(19) United States
(12) Patent Application Publication
(10) Pub. No.: US 2023/0347129 A1
(43) Pub. Date: Nov. 2, 2023

(54) ENTERAL CONNECTOR

(71) Applicant: Francis Palma Fabrigas, San Antonio, TX (US)

(72) Inventor: Francis Palma Fabrigas, San Antonio, TX (US)

(21) Appl. No.: 18/220,857

(22) Filed: Jul. 12, 2023

Related U.S. Application Data
(60) Provisional application No. 60/389,051, filed on Jun. 14, 2002.
(60) Provisional application No. 63/415,654, filed on Oct. 12, 2022.

(51) Int. Cl. A61M 39/10

(52) U.S. Cl. CPC A61M 39/105; A61M 2039/1077

(57) ABSTRACT

The present invention relates to an improved enteral connector having a valve port with an interconnected one-way air valve, a proximal port, a distal port, and a syringe port. In a proximal port block position, the one-way air valve is configured to allow ambient air to ingress into the improved enteral connector and interconnected syringe by way of the syringe port during removal of the syringe plunger abating the buildup of negative pressure within the improved enteral connector that would draw gastric contents from the enteral tube into an interconnected syringe, thus allowing the syringe to remain connected to the syringe port while the syringe plunger is removed to refill the syringe with treatment contents such as medicine, food, and other treatment contents during patient treatment.

### CROSS-REFERENCE TO RELATED APPLICATIONS

This application contains subject matter which is related to the subject matter of the following co-pending applications. The below-listed applications are hereby incorporated herein by reference in their entirety:
This is a U.S. non-provisional application that claims the benefit of U.S. provisional application, Ser. No. 60/389,051, inventor Francis Fabrigas, entitled “ENTERAL CONNECTOR”, filed Jul. 14, 2022; and
claims the benefit of U.S. provisional application, Ser. No. 63/415,654, inventor Francis Fabrigas, entitled, “ENTERAL TUBE”, filed Oct. 12, 2022.

### TECHNICAL FIELD OF THE INVENTION

This invention relates to an improved enteral connector having a valve port that houses a one-way air valve, a proximal port, a distal port, and a syringe port. In a proximal port block position, the one-way air valve is configured to allow ambient air to ingress into the improved enteral connector and interconnected syringe by way of the syringe port during removal of the syringe plunger abating the buildup of negative pressure within the connector that would otherwise draw gastric contents from the enteral tube into an interconnected syringe, thus allowing the syringe to remain connected to the syringe port while the syringe plunger is removed to refill the syringe with treatment contents such as medicine, food, and other treatment contents during patient treatment.

### BACKGROUND OF THE INVENTION

Enteral administration is known in the industry to administer formula, medication, and fluids to a patient through an enteral tube using a feeding pump or an enteral syringe. When a formula is delivered through a feeding pump, it is a practice to attach an enteral adapter or connector (usually a 3-way stopcock) to facilitate the administration of enteral liquid medication or medication mixture and water flushes using an enteral syringe particularly when the enteral tube is connected to a feeding bag. The enteral formula may also be administered directly into an enteral tube through an enteral connector using an enteral syringe during bolus feeding. Enteral connectors/adapters such as the commercially available ICU Medical's “LOPEZ VALVE” or the Dale's “ACE CONNECTOR” are provided with a port for an enteral syringe to be attached to deliver enteral medication or fluid.

While the above enteral connectors help during the administration of formula, medication, or water flush through an enteral tube, they require repeated manipulation of the enteral connector and/or repeated removal and reattachment of the enteral syringe from the port. When an enteral syringe is connected to the currently available 3-way connector, particularly when the prior enteral connector itself is connected proximally and distally to a feeding bag tube and the patient's enteral tube respectively, the clinician has to perform multiple steps during formula and medication administration such as repeated closing and opening of the port and repeated removal and/or reconnection of the enteral syringe from a port of the enteral connector.

When using currently available enteral connectors, in order to pour another batch of formula, medication, or fluid into the enteral syringe barrel (with its plunger in the barrel) that is connected to the enteral connector (with all ports in use), it is necessary to disconnect and reconnect the enteral syringe from the port to be able to retract and/or remove the enteral syringe plunger from the barrel (for filling and refilling) as it is difficult if not impossible to retract and remove the enteral syringe plunger out of the enteral syringe barrel without creating a vacuum or suctioning either gastric content or enteral formula. The required multiple steps of disconnection-reconnection of the enteral syringe from the enteral adapter port and/or the need to manipulate the enteral connector dial, to repeatedly close and open the connector port, makes enteral fluid administration cumbersome for the clinician, reducing workflow efficiency and increasing risk of errors. Clinicians are also subjected to an increased risk of body fluid exposure (e.g. gastric juices) every time the enteral syringe port is open during disconnection or at any time there is a need to manipulate the enteral connector dial.

The present invention addresses these and other shortcomings by providing an improved enteral connector. For these reasons and shortcomings as well as other reasons and shortcomings there is a long-felt need that gives rise to the present invention.

### SUMMARY OF THE INVENTION

The shortcomings of the prior art are overcome and additional advantages are provided through the provision of an improved enteral connector comprising a cylindrical body. The cylindrical body can comprise a valve port, a proximal port, a distal port, and a syringe port. The proximal port can be configured to interconnect with a feeding bag tube or a suction line. The distal port can be configured to interconnect with an enteral tube. The syringe port can be configured to interconnect with a syringe. The syringe can comprise a syringe plunger that is removable from the syringe.

Continuing, a one-way air valve interconnects with the valve port and is configured to allow ambient air to ingress into the cylindrical body through the one-way air valve abating the buildup of negative pressure within the cylindrical valve body. And, a columnar plug interconnects with the cylindrical body and can be rotated to at least a proximal port block position. The columnar plug has more than one flow hole that is interconnected. Each of the flow holes is perpendicular to the outer circumference of the columnar plug and extends inward interconnecting with other of the flow hole proximate to the center of the interior of the columnar plug. Each of the flow holes is positioned on the circumference of the columnar plug in a manner that when the columnar plug is rotated by a user, to each block position, fluid communication of air or liquid is allowed to pass through the flow holes between fewer than all of the following: the valve port, the proximal port, the distal port, and the syringe port.

The user rotates the columnar plug to select the proximal port block position where at least one of the flow holes is aligned with each of the syringe port, the distal port, and the valve port effectuating fluid communication of liquid between the syringe port and the distal port, and fluid communication of air between the valve port and the syringe port. The proximal port is absent one of the flow holes and blocked from fluid communication. In operation, the user secures the syringe to the syringe port. During removal of the syringe plunger from the syringe air ingresses through the one-way air valve into the cylindrical body abating the buildup of negative pressure that would otherwise draw gastric contents from the enteral tube into the syringe, thus allowing the syringe to remain connected to the syringe port while the syringe plunger is removed to refill the syringe with treatment contents during patient treatment.

Additional shortcomings of the prior art are overcome and additional advantages are provided through the provision of an improved enteral connector comprising a cylindrical body, a valve port interconnected on the circumference of the cylindrical body, and a one-way air valve that interconnects with the valve port and is configured to allow ambient air to ingress into the cylindrical body through the one-way air valve abating the buildup of negative pressure within the cylindrical valve body.

Continuing, the improved enteral connector can comprise a valve port cover that can be removed, and when secured seals the valve port closed preventing air from ingressing into the cylindrical body through the one-way air valve. A proximal port is interconnected on the circumference of the cylindrical body. The proximal port can be configured to interconnect with a feeding bag tube or a suction line. A distal port can be interconnected on the circumference of the cylindrical body. The distal port can be configured to interconnect with an enteral tube. A syringe port can be interconnected on the circumference of the cylindrical body. The syringe port can be configured to interconnect with a syringe. The syringe can comprise a syringe plunger that is removable from the syringe.

Continuing, the improved enteral connector can comprise a columnar plug that interconnects with the cylindrical body and is rotatable to at least a proximal port block position. The columnar plug has more than one flow hole that is interconnected. Each of the flow holes is perpendicular to the outer circumference of the columnar plug and extends inward interconnecting with other of the flow hole proximate to the center of the interior of the columnar plug. Each of the flow holes is positioned on the circumference of the columnar plug in a manner that when the columnar plug is rotated by a user, to each block position, fluid communication of air or liquid is allowed to pass through the flow holes between fewer than all of the following: the valve port, the proximal port, the distal port, and the syringe port.

The user rotates the columnar plug to select the proximal port block position where at least one of the flow holes can be aligned with each of the syringe port, the distal port, and the valve port effectuating fluid communication of liquid between the syringe port and the distal port, and fluid communication of air between the valve port and the syringe port. The proximal port is absent one of the flow holes and blocked from fluid communication. In operation, the user secures the syringe to the syringe port. During removal of the syringe plunger from the syringe air ingresses through the one-way air valve into the cylindrical body abating the buildup of negative pressure that would otherwise draw gastric contents from the enteral tube into the syringe, thus allowing the syringe to remain connected to the syringe port while the syringe plunger is removed to refill the syringe with treatment contents during patient treatment.

Additional shortcomings of the prior art are overcome and additional advantages are provided through the provision of a method of using an improved enteral connector. The method comprises the steps of attaching an enteral tube to a distal port of an improved enteral connector. The improved enteral connector comprises a cylindrical body that comprises a valve port, a proximal port, a distal port, and a syringe port. The proximal port is configured to interconnect with a feeding bag tube or a suction line. A one-way air valve interconnects with the valve port and is configured to allow ambient air to ingress into the cylindrical body through the one-way air valve abating the buildup of negative pressure within the cylindrical valve body. A columnar plug interconnects with the cylindrical body and is rotatable to at least a proximal port block position. The columnar plug has more than one flow hole that is interconnected. Each of the flow holes is perpendicular to the outer circumference of the columnar plug and extends inward interconnecting with other of the flow hole proximate to the center of the interior of the columnar plug. Each of the flow holes is positioned on the circumference of the columnar plug in a manner that when the columnar plug is rotated by a user, to each block position, fluid communication of air or liquid is allowed to pass through the flow holes between fewer than all of the following: the valve port, the proximal port, the distal port, and the syringe port.

The method continues by, connecting a syringe to the syringe port. The syringe comprises a syringe plunger that is removable from the syringe, and setting, by way of the user, the proximal port block position by rotating the columnar plug, where at least one of the flow holes is aligned with each of the syringe port, the distal port, and the valve port effectuating fluid communication of liquid between the syringe port and the distal port, and fluid communication of air between the valve port and the syringe port, the proximal port is absent one of the flow hole and blocked from fluid communication.

The method continues by, dispensing treatment contents from the syringe through the improved enteral connector and into the enteral tube, and removing the syringe plunger while the syringe is connected to the syringe port. During removal of the syringe plunger from the syringe air ingresses through the one-way air valve into the cylindrical body abating the buildup of negative pressure that would otherwise draw gastric contents from the enteral tube into the syringe, thus allowing the syringe to remain connected to the syringe port while the syringe plunger is removed. And, refilling the syringe with the contents, and then returning to the step of dispensing until treatment is complete. During the dispensing of the syringe contents into the enteral tube, the one-way valve prevents leakage of fluid from the enteral connector vent port.

Additional features and advantages are realized through the techniques of the present invention. Other embodiments and aspects of the invention are described in detail herein and are considered a part of the claimed invention. For a better understanding of the invention with advantages and features, refer to the description and the drawings.

### BRIEF DESCRIPTION OF THE FIGURES

The subject matter which is regarded as the invention is particularly pointed out and distinctly claimed in the claims at the conclusion of the specification. The foregoing and other objects, features, and advantages of the invention are apparent from the following detailed description taken in conjunction with the accompanying drawings in which:

FIG. 1 illustrates one example of an enteral connector;

FIG. 2 illustrates one example of a disassembled view of an enteral connector;

FIG. 3 illustrates one example of a front view of an enteral connector connected to a feeding pump tubing, an enteral tube, and an enteral syringe with the plunger in a semi-retracted position;

FIG. 4 illustrates one example of a cross-sectional view of an enteral connector;

FIG. 5 illustrates one example of a front view of an enteral connector connected to a feeding bag tubing, an enteral tube, and an enteral syringe with the enteral syringe plunger in a depressed position;

FIG. 6 illustrates one example of a cross-sectional view of an enteral connector;

FIG. 7 illustrates one example of a front view of an enteral connector connected to an enteral feeding bag tube and an enteral tube with the valve port and the enteral syringe port covers in place;

FIG. 8 illustrates one example of a cross-sectional view of an enteral connector;

FIG. 9 illustrates one example of an enteral tube;

FIG. 10 illustrates one example of a close-up elevational view of the proximal end of an enteral tube;

FIG. 11 illustrates one example of a top view of the proximal portion of an enteral tube;

FIG. 12 illustrates one example of a cross-sectional view of the proximal portion of an enteral tube;

FIG. 13 illustrates one example of a top view of an exemplary embodiment of an enteral tube that comprises a removable one-way air valve coupler that couples with the valve port;

FIG. 14 illustrates one example of a cross-sectional view of an exemplary embodiment of an enteral tube that comprises a removable one-way air valve coupler that couples with the valve port;

FIG. 15 illustrates one example of a top view of an exemplary embodiment of an enteral tube with a coupler comprising a one-way air valve connected to the valve port;

FIG. 16 illustrates one example of a cross-sectional view of an exemplary embodiment of an enteral tube with a coupler comprising a one-way air valve connected to the valve port;

FIG. 17 illustrates one example of an exemplary embodiment of an enteral tube connector;

FIG. 18 illustrates one example of a cross-sectional view of an exemplary embodiment of an enteral tube connector;

FIGS. 19-20 illustrate examples of exemplary embodiments of an enteral tube;

FIG. 21 illustrates one example of a coupler that comprises the one-way air valve;

FIG. 22 illustrates one example of a cross-sectional view of a coupler that comprises the one-way air valve;

FIG. 23 illustrates one example of a method of using an improved enteral connector; and

FIG. 24 illustrates exemplary embodiments that can be interchangeably used with the methods of the present invention.

The detailed description explains the preferred embodiments of the invention, together with advantages and features, by way of example with reference to the drawings.

### DETAILED DESCRIPTION OF THE INVENTION

The present invention relates to an enteral connector 200 in a 4-way stopcock configuration having four ports. An advantage, in the present invention, is that the enteral connector 200 has a one-way air valve 205 in one of the ports which allows a syringe to remain connected to the enteral connector 200 while the syringe plunger 232 is removed allowing the syringe 203 to be quickly reloaded with treatment content 504 and avoiding stomach contents from being drawn back into the syringe 230. In contrast, prior enteral connectors require the syringe to be removed from the enteral connector so that the plunger can be retracted or removed and the syringe reloaded. In prior enteral connectors, failure to remove the syringe from the enteral connectors when removing the syringe plunger can result in stomach contents being drawn back into the syringe. For disclosure purposes, stomach contents can be referred to as gastric contents, and such treatment contents 504 can be medications, food, water flush, or other contents as may be required and/or desired in a particular embodiment or patient treatment.

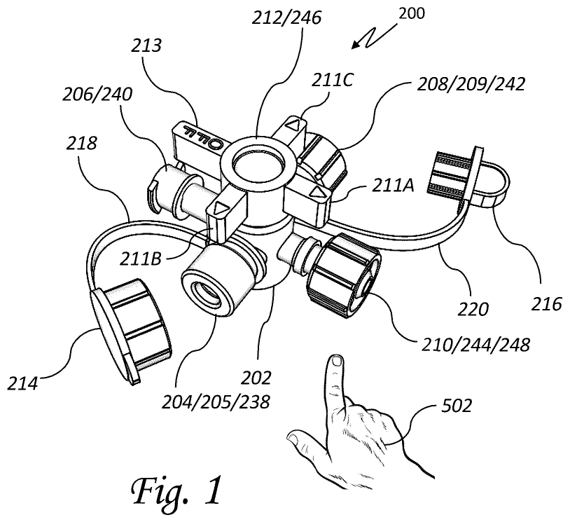

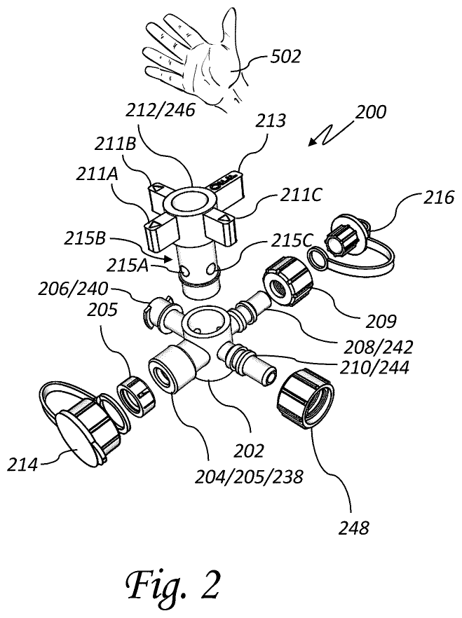

Turning now to the drawings in greater detail, it will be seen that in FIG. 1 there is illustrated one example of an enteral connector 200. In an exemplary embodiment, the enteral connector 200, of the present invention, can comprise a main cylindrical valve body 202 which has a plurality of flow holes 215 that interconnect within the cylindrical valve body 202 which enables a plurality of flow paths 238, 240, 242, 244 that are coupled to the flow holes 215 as better illustrated in at least FIGS. 2 and 4 to pass through the cylindrical valve body 202. A columnar plug 212 is inserted into the cylindrical valve body 202 and is rotatable relative to the cylindrical valve body 202.

The columnar plug 212 is configured with a T-shaped handle 246 that has finger-engageable protrusions 211A, 211B, 211C, and 213. Better illustrated in at least FIG. 2, the T-shaped finger-engageable protrusion 213 corresponds to the side of the cylindrical body 202 for which there is not a flow hole 215 and thus when the columnar plug 212 is rotated by a user 502 whichever port position 204, 206, 208, or 210 the T-shaped finger-engageable protrusion 213 points to has the flow block through the enteral connector 200. In this regard, finger-engageable protrusion 213 can also serve as a flow direction indicator with suitable markings to inform a user 502 of the enteral connector 200.

For disclosure purposes, a user 502 can be a medical person, patient, or other qualified-to-use people, as may be required and/or desired in a particular embodiment.

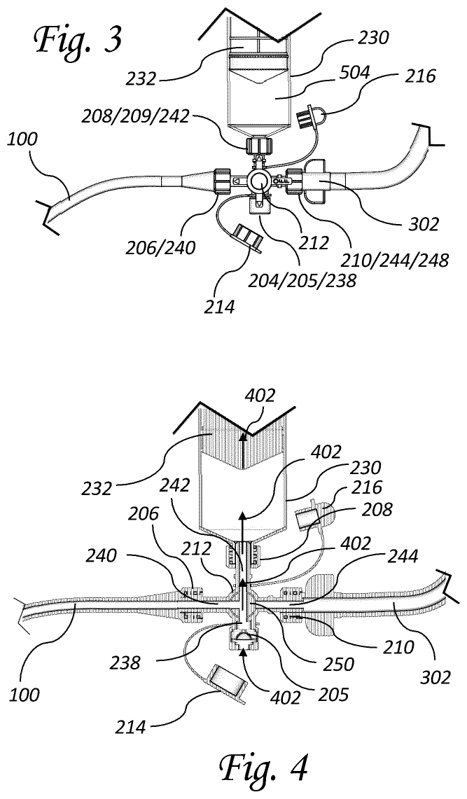

In operation, at least one flow path 238/240/242/244 can be blocked in a first columnar plug position, and when the columnar plug 212 is rotated by way of the T-shape handle to a second columnar position at least one of a different flow path 238/240/242/244 can be blocked. As an example illustrated in FIG. 1, in the first columnar plug 212 position port position 206 flow path 240 is blocked preventing contents from ingressing or egressing through the enteral tube 100. As illustrated in FIG. 2, in a second columnar plug position, port position 208 flow path 242 is blocked preventing contents from ingressing or egressing through the enteral connector from the syringe port 208 and thus from an interconnected syringe 230. As illustrated in FIG. 3, port position 210 flow path 244 is blocked preventing ingress or egress through the feeding bag tube 302.

In a plurality of exemplary embodiment, at least one of the flow paths 238, 240, 242, or 244 can be blocked preventing air or liquid flow through at least one of the flow holes 215A-C. In this regard, the flow state between the flow paths 238, 240, 242, and 244 can be enabled or blocked, as may be required and/or desired by user 502.

In operation, the t-shaped flow hole 215A-C creates a liquid or air connection between ports 204, 206, 208, and 210 and the columnar plug 212 can be rotated to selectively choose the desired port 204, 206, 208, or 210 interconnection pathway. Noting, that port 204 has a one-way air valve and port pathway 238 is an air pathway.

The columnar plug 212 defines a preferred form of central manifold that resides within valve body 202. Air or fluid can flow through each of the fluid pathways 238, 240, 242, or 244 embedded within the columnar plug 212 flow holes 215A-C. In operation, this flow happens when the flow holes 215A-C are aligned with the flow pathways 238, 240, 242, or 244. In this regard, any one of the 4 ports 204, 206, 208, or 210 is opened or closed and the fluid or air entering or exiting the stopcock enteral connector 200 is permitted or blocked so long as the port flow pathway 238, 240, 242, or 244 is open between one of the flow holes 215A-C is open.

In an exemplary embodiment, while the three ports 206, 208, and 210 can allow bi-directional fluid flow, port 204 is provided as a one-way air valve 205 that is configured to allow air to be drawn, from external the enteral connector 200, into the enteral connector 200 and through the flow pathway 244. This allows the syringe plunger 232 to be removed without closing the syringe port 208 and disconnecting the syringe 230. In operation, user 502 can attach syringe 230 to the syringe port 208, turn the columnar plug 212 to close port 210 and then remove and insert the syringe plunger 232 as needed to fill the syringe 230 and dispense the syringe 230 treatment contents 504 into the patient through enteral tube 100 without closing the syringe port 208 and disconnecting the syringe 230 in order to remove the plunger 232 and refill the syringe 230.

In an exemplary embodiment, the columnar plug 212 is provided with indicators 211A-C, and 213 to allow the user 502 to determine which ports are open and which are closed. Such indicator can be embossed or de-embossed in the columnar plug 212 as appropriate so that a user 502 can see the markings.

In an exemplary embodiment, while one port 204 contains a one-way air valve 205, the remaining ports 206, 208, and 210 are configured so that they can connect to other enteral devices such as syringe 230, enteral tube 100, and feeding bag tube 302. Port 204 containing the one-way air valve 205 is provided with a sealing valve port cover 214 to selectively open or close valve port 204 for suctioning gastric contents and to protect and keep the one-way air valve 205 clean.

The cylindrical valve body 202 can comprise a plurality of ports 204, 206, 208, 210, and other ports as may be required and/or desired in a particular embodiment. Also, the cylindrical valve body 202 can comprise a plurality flow path 238, 240, 242, 244, and other flow paths, and flow holes 215A-C, and other flow holes through the columnar plug 212 as may be required and/or desired in a particular embodiment. Various numbers of operating states can be provided to the stopcock by changing the number of ports associated with the valve body and changing the position of the end of the flow path within the columnar plug.

In an exemplary embodiment, the enteral connector 200 of the present invention can comprise an enteral syringe-connectable inlet valve, and a plurality of different kinds, types, or brands of medical grade one-way air valves. The sealing cover 118 can be optionally provided to cover the one-way air valve 205. Additionally, the one-way air valve can be disposed along any part or location on the enteral connector 200. The one-way air valve can be fabricated from a combination of different kinds of materials such as plastic, silicone, metal, rubber, or other materials.

In an exemplary embodiment, the one-way air valve 205 can be integrated with/connected to other enteral devices for enteral use and/or with other applications. Additionally, the one-way air valve 205 can be provided with a filtration element pre, post, or within the one-way air valve 205 system itself. Furthermore, the one-way air valve 205 can be attached or selectively coupled to one of the ports 204, 206, 208, or 210 of valve body 202 as may be required and/or desired in a particular embodiment.

In an exemplary embodiment, ports 204, 206, 208, and 210 can be disposed at different positions, orientations, or locations on the enteral connector 200. Additionally, ports 204, 206, 208, and 210 can be ENFIT compatible, legacy compatible, or modified differently to connect with other devices.

Advantages of the enteral connector 200 of the present invention and in contrast with prior devices include a port 204 with one-way air valve 205. The one-way air valve 205 when exposed to negative pressure within the flow pathway 238 caused by the removal motion of the syringe plunger 232, from the syringe 230, allows ambient air to enter the enteral connector 200. In operation, this allows the syringe plunger 232 to be removed without drawing liquid gastric contents from the enteral tube 100 into the syringe 230. Thus the syringe 230 doesn't need to be removed from the enteral connector in order to remove the syringe plunger 232 to refill the syringe 230. As a result, this reduces the number of times the syringe 230 needs to be disconnected and reconnected to the enteral connector 200 thus speeding the administration of enteral formula, medication, or water flush to the patient. The enteral connector 200, of the present invention, also enables a user 502 to be able to select various flow pathways and block other flow pathways by manipulation of the T-shaped handle 246 during administration of formula, medication, or water flush.

Similarly, associated methods of use are unique in that it allows user 502 to be more efficient by having fewer steps to perform during the administration of enteral feeding, medication, and water flushes, promoting clinician (user 502) safety by reducing the risk of body fluid exposure, and helps reduce the risk of errors by providing fewer steps during enteral feeding, medication, and water administration thru the enteral tube 100.

Advantages of the enteral connector 200 of the present invention and in contrast with prior devices include the use of a port 204 with one-way air valve 205, and a valve port cover 214 for valve port 204 with one-way air valve 205 to selectively allow or restrict the use of and airflow through the port 204, the one-way air valve 205, and flow pathway 238. In operation, the one-way air valve 205 is active (allows air to enter the cylindrical body 202 through flow pathway 238 and pass through one of the flow holes 215) when negative pressure is present inside the cylindrical body 202 resulting from the syringe plunger 232 being removed from the plunger 230.

Referring to FIG. 1, there is illustrated one example of an enteral connector 200. In an exemplary embodiment, the enteral connector 200 comprises a main cylindrical body 202 with four ports 204, 206, 208, and 210 that are integrally formed along the surface of the main body 202 at intervals in the circumferential direction. The ports 204, 206, 208, and 210 each have a flow pathway 238, 240, 242, and 244 respectively. An outer peripheral surface is supported by the inner peripheral surface of the cylindrical body 202. The enteral connector 200 also comprises a columnar plug 212 that is rotatable relative to the cylindrical body 202. The columnar plug 212 can be formed with a T-shaped handle 211A-C, and 213 and more than one flow hole 215A-C which are better illustrated in at least FIG. 2 so that when the columnar plug 212 is rotated, selectively at least one of the flow pathways 238, 240, 242, and 244 is blocked and the remaining flow paths are coupled together by way of the flow holes 215A-C.

In an exemplary embodiment, the flow holes 215A-C create a fluid/air connection between three of ports 204, 206, 208, and 210, and rotation of the T-shaped handle 211A-C, and 213 selects which of the ports are interconnected and which is port is blocked.

In an exemplary embodiment, the columnar plug 212 is provided with a fluid direction indicator dial 211A-C and 213 that can be manipulated to select which port to open or close. At least three of the four ports 204, 206, 208, and 210 depending on the columnar plug 112 position are in fluid communication by way of flow paths 238, 240, 242, and 242 and flow holes 215A-C inside of the enteral connector 200 wherein fluid communication between ports 204, 206, 208, and 210 are controlled by the columnar plug 212.

In an exemplary embodiment, the first port 204 also called the valve port 204 comprises a one-way air valve 205 to selectively allow external air to be drawn into the enteral connector 200 when negative pressure is present inside the enteral connector 200. At a 90-degree angle from the first port 204 is a second port 206 also called the distal port 206. The second port 206 can be configured as in a female ENFIT connector. At a 90-degree angle from the second port 206 can be a third port 208 also called the enteral syringe port 208. The third port 208 can be a male ENFIT configuration to receive an ENFIT compatible enteral syringe 230. At 90-degrees to the third port 208 can be a fourth port 210 also called the proximal port 210. The fourth port 210 can also be a male ENFIT configuration to receive the distal end of a feeding bag tubing 302. The valve port 204 and the enteral syringe port 208 are provided with sealing covers that can be referred to as the valve port cover 214 and the syringe port cover 216 respectively to selectively cover the valve port 204 and enteral syringe port 208 as needed. The fourth port 208 (proximal) can also be provided with a cover as desired and/or required in a particular embodiment. In operation, covering valve port 204 with valve port cover 214 can negate the ability of the one-way air valve 205 to allow air to ingress the enteral connector 200 thus allowing user 502 to configure valve port 204 to enable or disable the operation of one-way air valve 205, as desired or required, by removing the valve port cover 214 from the valve port 204 or sealing closed the valve port 204 with the valve port cover 214 respectively, such as when suctioning gastric contents to check for residuals and for other purposes. Furthermore, the syringe port cover 216 and be used selectively by user 502 to seal and unseal, for hygiene, leak, and other purposes, the enteral syringe port 208.

In an exemplary embodiment, in use, when an enteral feeding pump is used to administer enteral feeding, the proximal port 210 of the enteral connector 200 can be connected to the distal end of the enteral feeding pump tubing 302, and the distal port 206 of the enteral connector 200 can be connected to a patient's enteral tube 100. The t-shaped handle 211A-C and 213 are adjusted or otherwise aligned with the ports 204, 206, 208, and 210 so that the flow holes 215A-C corresponding to the T-shaped handle markings 211A-C of the columnar plug 212 create a fluid communication pathway between the proximal port 210, distal port 206, and valve port 204. The absence of a flow hole along the flow pathway 242 effectively closes the enteral syringe port 208. The valve port cover 214 can be kept in place sealing the valve port 204.

In an exemplary embodiment, when it is time to administer medications through the enteral connector 200, the enteral syringe port cover 216 is removed and an enteral syringe 230 is attached to the enteral syringe port 208. The enteral connector 200 T-shaped handle 211A-C and 213 is turned or otherwise aligned so that the columnar plug 212 flow holes 215A-C create fluid communication between flow pathways 238, 240, and 242 which are associated with the enteral syringe port 208, the valve port 204, and the distal port 206 (enteral tube 100 port). The valve port cover 214 and the syringe port cover 216 can be removed.

In operation, with the enteral syringe 203 in place connected to the syringe port 208, the plunger 232 can be removed from the enteral syringe 230 barrel to prepare for medication administration. In this regard, the mixture of medication can be poured into the barrel of syringe 230 while syringe 230 is attached to the syringe port 208 and the syringe plunger 232 can be inserted into the syringe 230 barrel and depressed to displace the treatment contents 504 through the enteral connector 200 and into the enteral tube 100. Once syringe 230 is empty, the syringe plunger 232 can be retracted and removed from the barrel of syringe 230. This happens by way of the one-way air valve allowing air to be drawn into the enteral connector 200 to counter the negative pressure the syringe plunger 232 creates as it is being removed. Once removed, syringe 230 can be refilled and the syringe plunger 232 reinserted in syringe 230 to dispense another batch of medication or fluids. This method is repeated until the treatment cycle is completed as necessary. When finished with dispensing medication and water flush, the t-shaped handle 211A-C and 213 can be repositioned or otherwise realigned such that fluid communication is restored between the proximal port 210 and distal port 206. The valve port cover 214 and the syringe port cover 216 can be fastened to seal the valve port 204 and syringe port 28 respectively.

In an exemplary embodiment, to use the enteral connector 200 for gastric emptying by manual aspiration or hooking to intermittent suction, the columnar plug 212 can be positioned so that there is a continuous flow pathway between the distal port 206 (the enteral tube 100 port), the proximal port 210 (connected to suction), and the valve port 204 and enteral syringe port 208 are sealed with their respective valve port cover 214 and syringe port cover 216 to maintain the negative pressure along the flow pathways 240 and 244 inside the enteral connector 200 created by a suction device.

Referring to FIG. 1, there is illustrated one example of an enteral connector 200. In an exemplary embodiment, the enteral connector 200 comprises a main cylindrical body 202, a plurality of ports including valve port 204, distal port 206, syringe port 208, and proximal port 210. Valve port 204 can be integrally formed on the enteral cylindrical body 202 and house a one-way air valve 205. Distal port 206 can be a female ENFIT connector and can be integrally formed on the enteral cylindrical body 202. Enteral syringe port 208 and proximal port 210 can be a male ENFIT connector and can be integrally formed on the enteral cylindrical body 202. A columnar plug 212 can be inserted inside the cylindrical body 202. A first cover 214 is provided for the valve port 204 and a second cover 216 is provided for the enteral syringe port 208. The covers 214, and 216 can be attached by a flexible strap 218, and 220 to the cylindrical body 202 proximate to their respective port 204/208.

Referring to FIG. 2, there is illustrated one example of a disassembled view of an enteral connector 200. Illustrated, in an exemplary embodiment, is how the cylindrical body 202 can be interconnected with the columnar plug 212, and how the T-shaped handle formed by 211A-C and 213 can be user 502 finger-engageable protrusions that also serve as flow direction indicators with handle protrusion 213 indicating by pointing and with the inscription to the port that is blocked, occluded, or otherwise turned ‘OFF’. The columnar plug 212 has flow holes 215A-C that direct the flow of fluid or air from one port to another through the columnar plug 212 by way of flow pathways 238, 240, 242, and/or 244. The valve port 204 has fastened therein a one-way air valve 205. The enteral syringe port 208 and proximal port 210 have threaded a rotatable collar 209, and 248 surrounding their respective ports 208, and 210 designed to engage to fasten to another compatible-ended enteral device.

In an exemplary embodiment, an improved enteral connector 200 can comprise a cylindrical body 212. The cylindrical body 212 can comprise a valve port 204, a proximal port 210, a distal port 206, and a syringe port 208. The proximal port 210 can be configured to interconnect with a feeding bag tube/suction line 302. The distal port 206 can be configured to interconnect with an enteral tube 100. The syringe port 208 can be configured to interconnect with a syringe 230. The syringe 230 can comprise a syringe plunger 232 that is removable from the syringe 230.

Continuing, a one-way air valve 205 interconnects with valve port 204 and is configured to allow ambient air to ingress into the cylindrical body 202 through the one-way air valve 205 abating the buildup of negative pressure within the cylindrical valve body 202. And, a columnar plug 212 interconnects with the cylindrical body 202 and can be rotated to at least a proximal port block position (better illustrated in at least FIG. 3). The columnar plug 212 has more than one flow hole 215A-C that is interconnected. Each of the flow holes 215A-C is perpendicular to the outer circumference of the columnar plug 212 and extends inward interconnecting with other of the flow holes 215 proximate to the center of the interior of the columnar plug 212. Each of the flow holes 215 can be positioned on the circumference of the columnar plug 212 in a manner that when the columnar plug 212 is rotated by a user 502, to each block position, fluid communication of air or liquid is allowed to pass through the flow holes 215 between fewer than all of the following: valve port 204, the proximal port 210, the distal port 206, and the syringe port 208.

In an exemplary embodiment and for disclosure purposes, block position refers to the columnar plug 212 being positioned by user 502 to block at least one of the ports 204, 206, 208, or 210. In this regard, the block positions can include the proximal port block position, the distal port block position, the syringe port block position, and the valve port block position.

In the proximal port block position, the proximal port 210 is blocked and the valve port 204, the distal port 206, and the syringe port 208 are interconnected in a fluid communication (air/liquid) manner by flow holes 215A-C. The handle protrusion 213 faces the proximal port 210.

In the distal port block position, the distal port 206 is blocked and the valve port 204, the proximal port 210, and the syringe port 208 are interconnected in a fluid communication (air/liquid) manner by flow holes 215A-C. The handle protrusion 213 faces the distal port 206.

In the syringe port block position, the syringe port 208 is blocked and the valve port 204, the proximal port 210, and the distal port 206 are interconnected in a fluid communication (air/liquid) manner by flow holes 215A-C. The handle protrusion 213 faces the syringe port 208.

In the valve port block position, the valve port 204 is blocked and the syringe port 208, the proximal port 210, and the distal port 206 are interconnected in a fluid communication manner by flow holes 215A-C. The handle protrusion 213 faces valve port 204.

In operation, user 502 rotates the columnar plug 212 to select the proximal port block position (which is better illustrated in at least FIG. 3) where at least one of the flow hole 215 is aligned with each of the syringe port 208, the distal port 206, and the valve port 204 effectuating fluid communication of liquid between the syringe port 208 and the distal port 206, and fluid communication of air between the valve port 204 and the syringe port 208. The proximal port 210 is absent one of the flow holes 215 and blocked from fluid communication. User 502 secures syringe 230 to syringe port 208. During the removal of the syringe, plunger 232 from the syringe 230 air ingresses through the one-way air valve 205 into the cylindrical body 202 abating the buildup of negative pressure that would otherwise draw gastric contents from the enteral tube 100 into the syringe 230, thus allowing the syringe 230 to remain connected to the syringe port 208 while the syringe plunger 232 is removed to refill the syringe 230 with treatment contents 504 during treatment. Such treatment contents 504 can be medications, food, water flush, or other contents as may be required and/or desired in a particular embodiment or patient treatment.

In an exemplary embodiment, a valve port cover 214 can be removable, and when secured seals the valve port 204 closed preventing air from ingressing into the cylindrical body 202 through the one-way air valve 205.

In an exemplary embodiment, in operation, user 502 rotates the columnar plug 212 to select a syringe port block position (better illustrated in at least FIG. 2) where at least one of the flow holes 215 is aligned with the proximal port 210, the distal port 206, and the valve port 204 effectuating fluid communication between the proximal port 210 and the distal port 206. The syringe port 208 is absent one of the flow holes 215 and blocked from fluid communication. In treatment when the suction line 302 is interconnected with the proximal port 210 the user 502 secures the valve port cover 214 to the valve port 204 preventing air from ingressing into the cylindrical body 202 to maintain suction within the cylindrical body 202 during treatment.

In an exemplary embodiment, in operation, user 502 rotates the columnar plug 212 to select a distal port block position (which is better illustrated in at least FIG. 1) where at least one of the flow holes 215 are aligned with each of the syringe port 208, the proximal port 210, and the valve port 204 effectuating fluid communication of liquid between the syringe port 208 and the proximal port 210, and fluid communication of air between the valve port 204 and the syringe port 208. The distal port 206 is absent one of the flow holes 215 and blocked from fluid communication. User 502 secures syringe 230 to syringe port 208. During the removal of the syringe plunger 232 from the syringe 230 air ingresses through the one-way air valve 205 into the cylindrical body 202 abating the buildup of negative pressure that would otherwise draw gastric contents from the feeding bag tube/suction line 302 into the syringe 230, thus allowing the syringe 230 to remain connected to the syringe port 208 while the syringe plunger 232 is removed to refill the syringe 230 with treatment contents 504 during treatment.

In an exemplary embodiment, each of the valve port 204, proximal port 210, syringe port 208, and distal port 206 can be positioned at 90-degree increments around the circumference of the cylindrical body 212.

In an exemplary embodiment, each of the proximal port 210, distal port 206, and syringe port 208 are ENFIT compatible.

In an exemplary embodiment, a T-shaped handle 246 can comprise a plurality of protrusions 211A-C and 213, when the columnar plug 212 is rotated to the proximal port block position (illustrated in at least FIG. 3) the plurality of protrusions are aligned with the valve port 204, proximal port 210, distal port 206, and the syringe port 208. The plurality of protrusions 211A-C and 213 comprise indicia that in the proximal port block position indicate the proximal port 210 is off or blocked (protrusion 213) and the distal port 206, the syringe port 208, and the valve port 204 are open or unblocked (protrusions 211A-C).

In an exemplary embodiment, the T-shaped handle 246 can be integrally formed on the columnar plug 212.

In an exemplary embodiment, a syringe port cover 216 can be removable, and when secured seals the syringe port 208 closed.

Referring to FIG. 3, there is illustrated one example of a front view of an enteral connector 200 connected to a feeding pump tubing 302, an enteral tube 100, and an enteral syringe 230 with the plunger 232 in a semi-retracted position. In an exemplary embodiment, the proximal port 210 is connected to a feeding bag tubing 302 and the distal port 206 is connected to a patient's feeding bag tube 100 with both covers 214, and 216 unfastened to their respective ports 204, and 208. Additionally, an enteral syringe 230 is connected to the enteral syringe port 208. The valve port 204, distal port 206, and enteral syringe port 208 are open while the proximal port 210 is closed as indicated by the columnar plug ‘OFF’ protrusion indicator 213 that is directed towards the proximal port 210.

In an exemplary embodiment, an improved enteral connector 200 can comprise a cylindrical body 202, a valve port 204 interconnected on the circumference of the cylindrical body 212, and a one-way air valve 205 that interconnects with the valve port 204 and is configured to allow ambient air to ingress into the cylindrical body 202 through the one-way air valve 205 abating buildup of negative pressure within the cylindrical valve body 202.

Continuing, the improved enteral connector 200 can comprise a valve port cover 214 that can be removed, and when secured seals the valve port 204 closed preventing air from ingressing into the cylindrical body 202 through the one-way air valve 205. A proximal port 210 is interconnected on the circumference of the cylindrical body 212. The proximal port 210 can be configured to interconnect with a feeding bag tube/suction line 302. A distal port 206 can be interconnected on the circumference of the cylindrical body 212. The distal port 206 can be configured to interconnect with an enteral tube 100. A syringe port 208 can be interconnected on the circumference of the cylindrical body 212. The syringe port 208 can be configured to interconnect with a syringe 230. The syringe 230 can comprise a syringe plunger 232 that is removable from the syringe 230.

Continuing, the improved enteral connector 200 can comprise a columnar plug 212 that interconnects with the cylindrical body 202 and is rotatable to at least a proximal port block position (better illustrated in at least FIG. 3). The columnar plug 212 has more than one flow hole 215A-C that is interconnected. Each of the flow holes 215A-C is perpendicular to the outer circumference of the columnar plug 212 and extends inward interconnecting with other of the flow holes 215 proximate to the center of the interior of the columnar plug 212. Each of the flow holes 215 can be positioned on the circumference of the columnar plug 212 in a manner that when the columnar plug 212 is rotated by a user 502, to each block position, fluid communication of air or liquid is allowed to pass through the flow holes 215 between fewer than all of the following: valve port 204, the proximal port 210, the distal port 206, and the syringe port 208. In operation, user 502 rotates the columnar plug 212 to select the proximal port block position where at least one of the flow holes 215A-C can be aligned with each of the syringe port 208, the distal port 206, and the valve port 204 effectuating fluid communication of liquid between the syringe port 208 and the distal port 206, and fluid communication of air between the valve port 204 and the syringe port 208. The proximal port 210 is absent one of the flow holes 215 and blocked from fluid communication. User 502 secures syringe 230 to syringe port 208. During the removal of the syringe, plunger 232 from the syringe 230 air ingresses through the one-way air valve 205 into the cylindrical body 202 abating the buildup of negative pressure that would otherwise draw gastric contents from the enteral tube 100 into the syringe 230, thus allowing the syringe 230 to remain connected to the syringe port 208 while the syringe plunger 232 is removed to refill the syringe 230 with treatment contents 504 during treatment.

Referring to FIG. 4, there is illustrated one example of a cross-sectional view of an enteral connector 200 showing the columnar plug 212 positioned with protrusions 211A-C indicating the flow pathways 238, 240, and 242 of the ports 204, 206, and 208 are open and the absence of a flow hole, stop 250 blocks the flow pathway 244 effectively blocking ‘OFF’ the proximal port 210. An enteral syringe 230 assembly is connected to the enteral syringe port 208 wherein the enteral syringe plunger 232 can be inserted and retracted from the barrel of the syringe 230. In operation, indicated by arrow 402, with a higher resistance from the distal enteral tube port 206 fluid pathway 240 and the valve port cover 214 removed, as the syringe plunger 232 is removed from the syringe 230 negative pressure is generated within the enteral connector 200. In this regard, the one-way air valve 205 opens up allowing air (as shown by arrow 402) to enter and pass through the enteral connector 200 and into the enteral syringe 230 to eliminate the negative pressure the motion of removing the syringe plunger 232 created thereby allowing the enteral syringe plunger 232 to be easily removed out of the barrel of the enteral syringe 230 without having to disconnect the syringe 230 from the enteral connector 200.

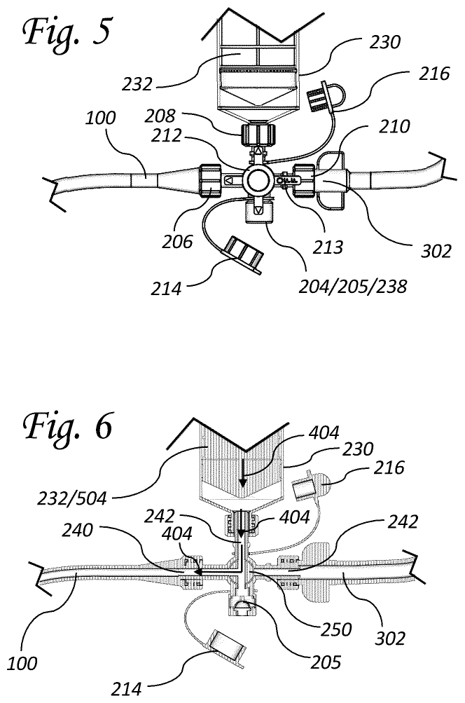

Referring to FIG. 5, there is illustrated one example of a front view of an enteral connector 200 connected to a feeding bag tubing 302, an enteral tube 100, and an enteral syringe 230 with the enteral syringe plunger 232 in a depressed position. In an exemplary embodiment, the enteral connector 200 the proximal port 210 can be coupled to a feeding bag tubing 302 (feeding bag not shown) and the distal port 206 can be coupled to a feeding bag tube 100 respectively with the proximal port 210 closed as indicated by the columnar plug 212 ‘OFF’ protrusion 213 position. An enteral syringe 230 can be connected to the enteral syringe port 208.

Referring to FIG. 6, there is illustrated one example of a cross-sectional view of an enteral connector 200 (with the T-shaped handle 211A-C and 213 and columnar plug 212 positioned as in FIG. 5). In an exemplary embodiment, in this configuration, when the syringe plunger 232 is being pushed 404 towards the end of the barrel as indicated by the arrows, the treatment contents 504 of the enteral syringe 230 are displaced and expelled towards the enteral connector 200 syringe port 208 flow pathway 242 then to the distal port 206 flow pathway 240 by way of the flow holes 215 and into the enteral tube 100 as the one-way air valve 205 remains closed (even when the valve cover 214 is open) in a neutral position during this time preventing leakage of contents. The absence of a flow hole 215 effectively stops 250 the flow of the contents through the proximal port 210 and into the feeding bag tube 302.

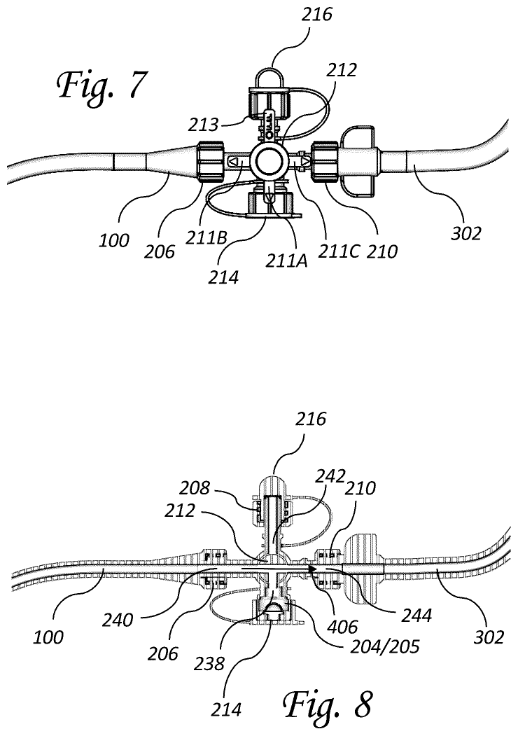

Referring to FIG. 7, there is illustrated one example of a front view of an enteral connector 200 connected to an enteral feeding bag tube 302 and an enteral tube 100 with the valve port cover 214 and the enteral syringe port cover 216 secured in place. Additionally, the columnar plug 212 is illustrated with the T-shaped handle ‘OFF’ protrusion 213 block or otherwise closing the enteral syringe port 208 with the t-shaped handle protrusions 211A-C illustrated that the proximal port 210, distal port 206 and valve port 204 are in the open position.

Referring to FIG. 8, there is illustrated one example of a cross-sectional view of an enteral connector 200 (with the T-shaped handle 211A-C and 213 and columnar plug 212 positioned as in FIG. 7). In an exemplary embodiment, when negative pressure such as during suctioning is generated on the feeding bag tube 302 port 210 and the columnar plug 212 positions in a manner to create a flow pathway 240/244 path between the distal port 206 and the proximal port 210, and the valve port cover 214 and the enteral syringe port cover 216 are secured in place to maintain the negative pressure during suctioning the fluid direction is indicated by the arrow 406 from the distal port 206 towards the proximal port 210.

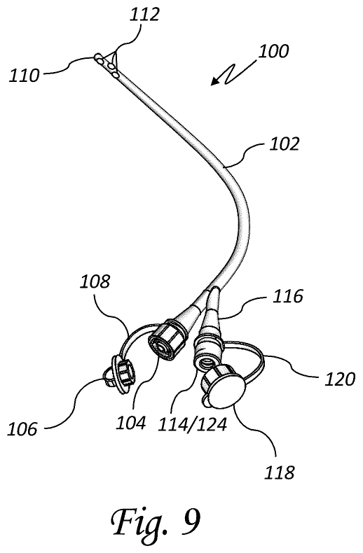

Referring to FIGS. 9-22, there is illustrated one example of an enteral tube 100. When contrasted to prior enteral tubes the enteral tube 100 of the present invention includes the use of a valve port 114 with an incorporated one-way air valve 124. In operation, the advantage is that the ambient air can enter the enteral tube 100 through the one-way air valve 124 to abate the buildup of negative pressure that can interfere with the flow operation within the enteral tube 100 when objects or apparatus such as enteral syringes are used. The one-way air valve allows such devices and apparatus to be used without further manipulation of the tubing system. With regards to enteral syringe use this use of the one-way air valve 124 to abate negative pressure such as when a syringe plunger is removed from the enteral syringe minimizes the number of disconnection-reconnection of the enteral syringe from a feeding port of the enteral tube 100 during enteral feeding administration.

Similarly, the benefits of the use of the enteral tube 100 include 1) allows user 502 to be more efficient by allowing less manipulation of the enteral tubing 100 system during the administration of enteral feeding, medication, and water flushes; 2) promotes clinician safety by reducing the risk of body fluid exposure; and 3) helps reduce the risk of errors by providing fewer steps during enteral feeding, medication, and water administration thru the enteral tube.

The enteral tube is structurally different from prior enteral tubes. More specifically, the enteral tube 100 of the present invention includes 1) a means that allows one-way entry of air into the inner diameter of an enteral tubing 100; 2) a cover 118 for the valve port 114 with one-way air valve 124 to selectively allow or restrict air to enter through the valve port 114 with one-way air valve 124; 3) a one-way air valve 124 that is activated (allows air to enter) when negative pressure is applied from the inside an enteral tube 100; and 4) has a plurality of fluid pathways one of which is a valve port 114 with one-way air valve 124.

In an exemplary embodiment, an enteral tube 100 can have a one-way air valve 124 positioned in valve port 114. In this regard, the enteral tube 100 can comprise a flexible elongated tubular body 102 with proximal and distal 110 ends. At the proximal end, there can be a proximal port 104 and a valve port 114. The valve-port 114 can comprise a one-way air valve 124. The proximal port 104 can be interconnected with the enteral connector 200 or in applications that don't use the enteral connector 200 the proximal port 104 can be connected to a feeding bag tube 302 or other compatible ENFIT device. For disclosure purposes, the proximal port 104 can also be referred to as the feeding port 104.

In an exemplary embodiment, the enteral tube 100 can be fabricated from flexible or rigid materials such as plastic, silicone, metal, rubber, or other materials as may be required and/or desired in a particular embodiment. Provided on the distal end 110 of the tubular body 102 are a plurality of holes 112 that are in fluid (air/liquid) communication with the valve port 114 and the proximal port 104. The valve port 114 comprises a one-way air valve 124 that allows ambient air to enter the tubular duct 116 and is disposed to the tubular body 102 preferably in a Y-shaped configuration.

The valve port 114 can also comprise a valve cover 118 that can be opened and sealed closed over the one-way air valve 124 as needed such as during aspiration of gastric contents and when the enteral tube 100 is connected to suction for gastric decompression and/or gastric emptying.

Advantages, in the present invention, of the enteral tube 100 include an enteral syringe-connectable valve body and interoperability with a plurality of different types and manufacturers of one-way air valves. Additionally, valve port 114 can be disposed at different positions or locations along the enteral tube 100, and valve port 114 can include a valve cover 118. Furthermore, the valve port 114 can be integrated with or couplable to other types of enteral devices for enteral use and/or with other applications.

In an exemplary embodiment, the enteral tube 100 can be any kind of enteral tube such as a Nasogastric Tube (NGT), Percutaneous Endoscopic Gastrostomy (PEG) tube, Jejunostomy tube (JT), or other types and/or kinds of enteral tubes as may be required and/or desired in a particular embodiment.

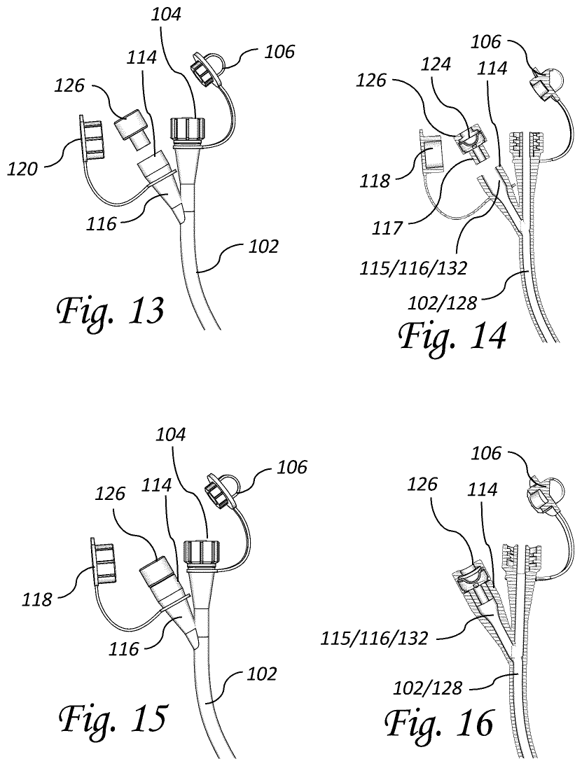

Other advantages, in the present invention, are that the proximal port 104 can be ENFIT compatible, legacy compatible, or modified differently to connect with other devices, and has a valve port 114 that can be provided with a filtration element pre, post, or within the one-way air valve 124 itself. As illustrated in at least FIG. 13, valve port 114 can be configured to selectively couple to one end of a valve port coupler 126 and on the other end to other enteral devices. In this regard, in an exemplary embodiment, the one-way air valve 124 can be integrated into the valve port coupler 126, and selectively, the valve port coupler 126 can be configured to interconnect with other enteral devices.

Referring to FIG. 9, there is illustrated one example of an enteral tube 100. In an exemplary embodiment, a tubular body 102 has proximal and distal end 110. At the proximal end is a proximal port 104 which can be configured as an ENFIT compatible male connector and the distal end 110 comprises a plurality of openings 112. Adjacent to the proximal port 104 is a branch from the tubular body 102 forming a valve port 114. The valve port 114 comprises a one-way air valve 124. The proximal port 104, which can be interconnected with a feeding bag tube connected from a feeding bag tube, comprises a removable proximal port cover 106 that can be attached to the neck of the proximal port 104 by a strip of flexible material 108. In a similar manner, valve port 114 can comprise a removable valve cover 118 that is attached to the valve port neck 116 by a strip of flexible material 120. The distal end 110 of the said enteral tubing has a plurality of holes 112 serving as outlets to allow content to traverse between the holes 112 and the proximal port 104 and valve port 114. In this regard, the proximal port 104, and valve port 114 are in fluid (air/liquid) communication with the plurality of holes 112 that are located proximate to the distal end 110.

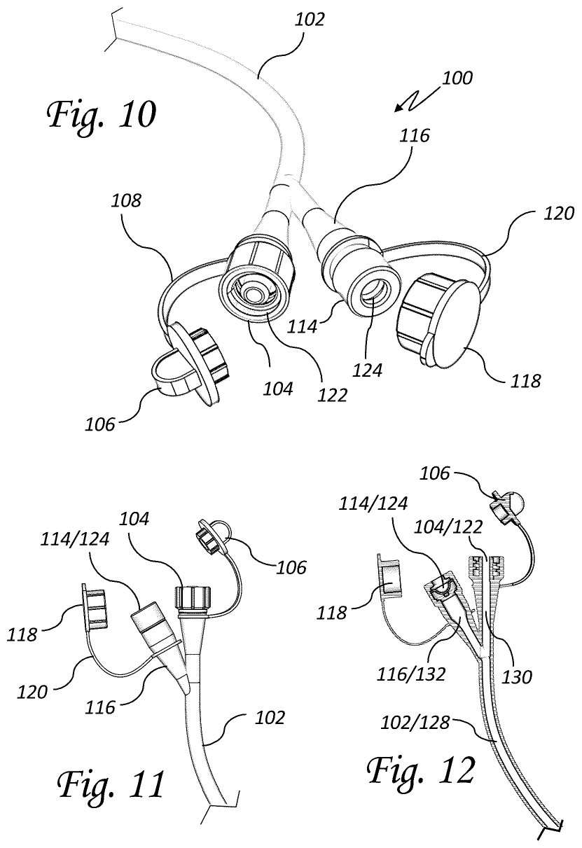

Referring to FIG. 10, there is illustrated one example of a close-up elevational view of the proximal end of an enteral tube 100. In an exemplary embodiment, the enteral tube 100 proximal port 104 has threads 122 on the inner surface that are connectable to ENFIT compatible devices.

Referring to FIGS. 11 and 12, there is illustrated one example of a top view of the proximal portion of an enteral tube 100. FIG. 12 is a cross-sectional view of the proximal portion of an enteral tube 100 which is illustrated in FIG. 11. In an exemplary embodiment, the one-way air valve 124 and how the flow pathway ducts 128, 130, and 132 are in fluid communication with each other.

Referring to FIGS. 13 and 14, there is illustrated one example of a top view of an exemplary embodiment of an enteral tube 100 with a one-way air valve coupler 126 that couples with the valve port. FIG. 14 is a cross-sectional view of FIG. 13. In an exemplary embodiment, one-way air valve coupler 126 can be removably coupled, and detached from the valve port 114. FIG. 13 illustrates how the distal and connecting end 117 of valve port 114 can be configured to receive the one-way air valve coupler 126 within the inner diameter 115 of the valve port 114 opening. The valve port 114 opening can be ENFIT compatible and the distal end 117 of the one-way air valve coupler 126 can be an ENFIT tip. In an exemplary embodiment, the one-way air valve coupler 126 can be configured to have a female ENFIT connector tip and the valve port 114 can be configured to be a male ENFIT port.

Referring to FIGS. 15 and 16, there is illustrated one example of an exemplary embodiment of an enteral tube 100 with a coupler 126 comprising a one-way air valve 124 connected to valve port 114. In this regard, FIG. 15 is the same embodiment of FIG. 13 with the valve-containing port 126 connected to valve port 114 and shown in FIG. 16 in its cross-sectional view.

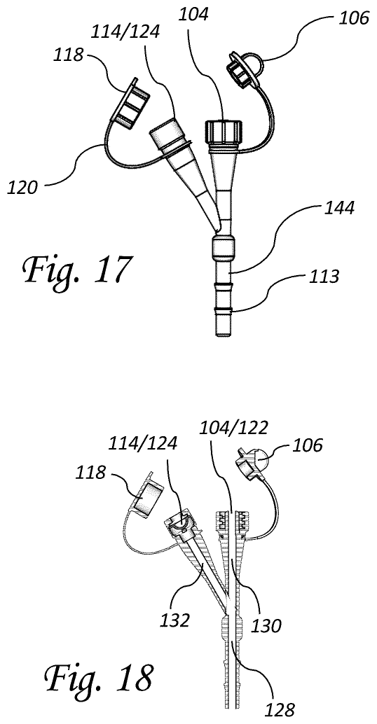

Referring to FIGS. 17 and 18, there is illustrated one example of an exemplary embodiment of an enteral tube 100 connector. FIG. 18 is a cross-sectional view of FIG. 17. In an exemplary embodiment, the enteral tube 100 can comprise a short primary tubing 144 of rigid material and ring bumps 113 to increase friction against the inner diameter of a coupled device and with the proximal port 104 and the valve port 114. FIG. 18 illustrates how the flow pathway ducts 130, 128, and 132 are in fluid (air/liquid) communication with each other.

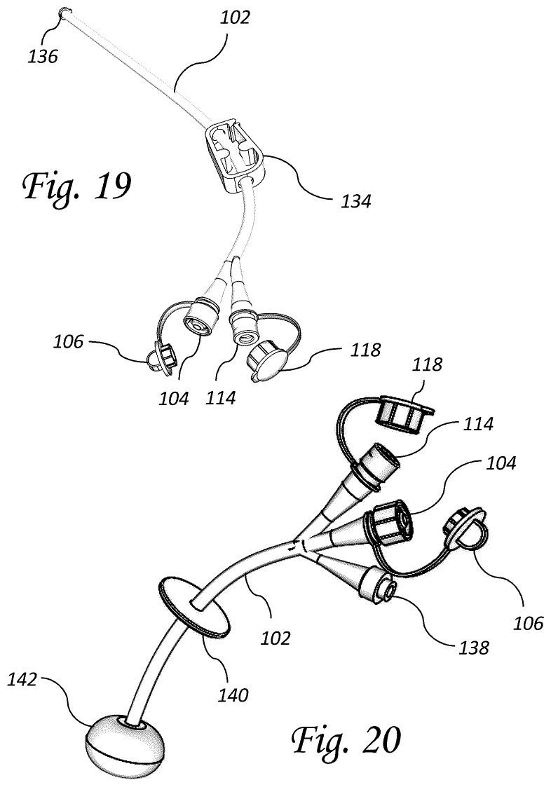

Referring to FIGS. 19 and 20, there are illustrated exemplary embodiments of the enteral tube 100. In an exemplary embodiment in FIG. 19, there is illustrated one example of the enteral tube 100 comprising a flexible main tubing 102 as well as having a connectable locking tip 136 for connection to low-profile enteral tubes such as MIC tube and a clamp 134 for flow control and with the same proximal openings of proximal port 104 and valve port 114.

In an exemplary embodiment in FIG. 20, there is illustrates one example of an enteral tube 100 comprising a flexible main tube 102, a disc stabilizer 140, and having an anchoring-element inflatable balloon 142 disposed at the distal end with 3 proximal ports which are the one-way valve port 114, the proximal port 104, and the balloon-inflation port 138.

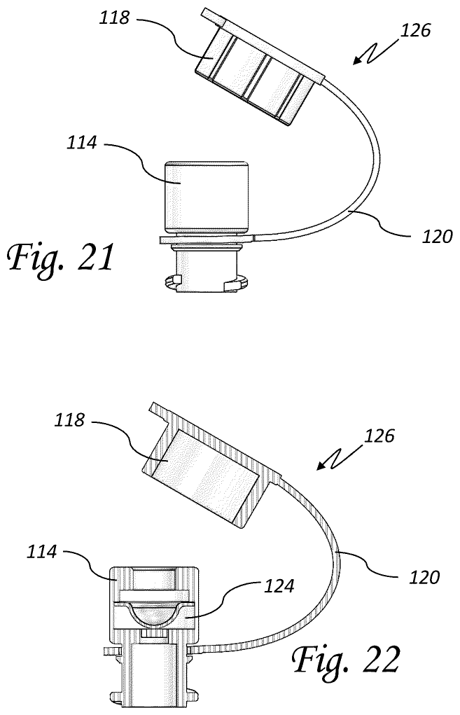

Referring to FIGS. 21 and 22, there is illustrated one example of a one-way valve coupler 126. FIG. 22 is a cross-sectional view of FIG. 21. In an exemplary embodiment, the one way valve coupler 126 (also illustrated in at least FIGS. 14 and 15) can have an ENFIT connectable tip 150 for interconnection with other ENFIT enteral devices.

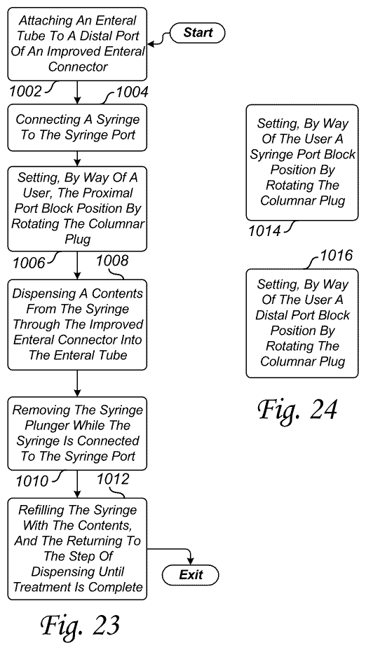

Referring to FIG. 23, there is illustrated one example of a method of using an improved enteral connector 200. The method begins in step 1002.

In step 1002, an enteral tube 100 can be attached to a distal port 206 of an improved enteral connector 200. The improved enteral connector 200 comprises a cylindrical body 202. The cylindrical body 202 can comprise a valve port 204, a proximal port 210, the distal port 206, and a syringe port 208. The proximal port 208 is configured to interconnect with a feeding bag tube/suction line 302. A one-way air valve 205 interconnects within the valve port 204 and is configured to allow ambient air to ingress into the cylindrical body 202 through the one-way air valve 205 abating the buildup of negative pressure within the cylindrical valve body 202. A columnar plug 212 interconnects with the cylindrical body 202 and is rotatable to at least a proximal port block position (better illustrated in at least FIG. 3). The columnar plug 212 has more than one flow hole 215A-C that is interconnected. Each of the flow holes 215A-C is perpendicular to the outer circumference of the columnar plug 212 and extends inward interconnecting with other of the flow holes 215 proximate to the center of the interior of the columnar plug 212. Each of the flow holes 215 can be positioned on the circumference of the columnar plug 212 in a manner that when the columnar plug 212 is rotated by a user 502, to each block position, fluid communication of air or liquid is allowed to pass through the flow holes 215 between fewer than all of the following: the valve port 204, the proximal port 210, the distal port 206, and the syringe port 208. The method then moves to step 1004.

In step 1004, syringe 230 is connected to syringe port 208. The syringe 230 comprises a syringe plunger 232 that is removable from the syringe 230. The method then moves to step 1006.

In step 1006, by way of user 502, the proximal port block position (better illustrated in at least FIG. 3) is set by rotating the columnar plug 212, where at least one of the flow holes 215 A-C is aligned with each of the syringe port 208, the distal port 206, and the valve port 204 effectuating fluid communication of liquid between the syringe port 208 and the distal port 206, and fluid communication of air between the valve port 204 and the syringe port 208. The proximal port 210 is absent one of the flow holes 215 and blocked from fluid communication. The method then moves to step 1008,

In step 1008, treatment contents 504 are dispensed from the syringe 230 through the improved enteral connector 200 and into the enteral tube 100. Such treatment contents 504 can be medications, food, water flush, or other contents as may be required and/or desired in a particular embodiment or patient treatment. The method then moves to step 1010.

In step 1010, the syringe plunger 232 is removed while syringe 230 is connected to the syringe port 208. During the removal of the syringe, plunger 232 from the syringe 230 air ingresses through the one-way air valve 205 into the cylindrical body 202 abating the buildup of negative pressure that would otherwise draw gastric contents from the enteral tube 100 into the syringe 230, thus allowing the syringe 230 to remain connected to the syringe port 208 while the syringe plunger 232 is removed. The method then moves to step 1012.

In step 1012, the syringe 230 can be refilled with the treatment contents 504, and then the method returns to step 1008 the step of dispensing until treatment is complete. In this regard, the syringe 230 can be connected to the syringe port 208 once and the syringe is repeatedly filled and dispensed into the patient by removal of the plunger 232 without disconnecting the syringe 230 from the syringe port 208.

Referring to FIG. 24, there are illustrated exemplary embodiments that can be interchangeably used with the methods of the present invention.

In step 1014, by way of user 502, a syringe port block position (better illustrated in at least FIG. 2) is set by rotating the columnar plug 212, where at least one of the flow holes 215A-C is aligned with the proximal port 210, the distal port 206, and the valve port 204 effectuating fluid communication between the proximal port 210 and the distal port 206. The syringe port 208 is absent one of the flow holes 215 and blocked from fluid communication. In treatment when the suction line 302 is interconnected with the proximal port 210 the user 502 can secure the valve port cover 214 to the valve port 204 preventing air from ingressing into the cylindrical body 202 to maintain suction within the cylindrical body 202 during treatment.

In step 1016, by way of user 502, a distal port block position (better illustrated in at least FIG. 1) is set by rotating the columnar plug 212, where at least one of the flow holes 215A-C is aligned with each of the syringe port 208, the proximal port 210, and the valve port 204 effectuating fluid communication of liquid between the syringe port 208 and the proximal port 210, and fluid communication of air between the valve port 204 and the syringe port 208. The distal port 206 is absent one of the flow holes 215 and blocked from fluid communication. User 502 can secure the syringe port cover 216 to the syringe port 208. During removal of the syringe, plunger 232 from syringe 230 air ingresses through the one-way air valve 205 into the cylindrical body 202 abating the buildup of negative pressure that would otherwise draw feeding bag tube contents from the feeding bag tube 302 into the syringe 230, thus allowing the syringe 230 to remain connected to the syringe port 208 while the syringe plunger 232 is removed to refill the syringe 230 with the treatment contents 504 during treatment.

The flow diagrams depicted herein are just examples. There may be many variations to these diagrams or the steps (or operations) described therein without departing from the spirit of the invention. For instance, the steps may be performed in a differing order, or steps may be added, deleted, or modified. All of these variations are considered a part of the claimed invention.

While the preferred embodiment of the invention has been described, it will be understood that those skilled in the art, both now and in the future, may make various improvements and enhancements which fall within the scope of the claims which follow. These claims should be construed to maintain the proper protection for the invention first described.

What is claimed is:

1. An improved enteral connector comprising:
a cylindrical body that comprises a valve port, a proximal port, a distal port, and a syringe port, the proximal port is configured to interconnect with a feeding bag tube or a suction line, the distal port is configured to interconnect with an enteral tube, the syringe port is configured to interconnect with a syringe, the syringe comprises a syringe plunger is removable from the syringe;
a one-way air valve interconnects with the valve port and is configured to allow ambient air to ingress into the cylindrical body through the one-way air valve abating buildup of negative pressure within the cylindrical valve body; and
a columnar plug interconnects with the cylindrical body and is rotatable to at least a proximal port block position, the columnar plug having more than one of a flow hole that are interconnected, each of the flow hole is perpendicular to outer circumference of the columnar plug and extends inward interconnecting with other of the flow hole proximate to center of interior of the columnar plug, each of the flow hole are positioned on circumference of the columnar plug in manner that when the columnar plug is rotated by a user, to each block position, fluid communication of air or liquid is allowed to pass through the flow holes between fewer than all of the following: the valve port, the proximal port, the distal port, and the syringe port, the user rotates the columnar plug to select the proximal port block position at least one of the flow hole is aligned with each of the syringe port, the distal port, and the valve port effectuating fluid communication of liquid between the syringe port and the distal port, and fluid communication of air between the valve port and the syringe port, the proximal port is absent one of the flow hole and blocked from fluid communication, in operation the user secures the syringe to the syringe port, during removal of the syringe plunger from the syringe air ingresses through the one-way air valve into the cylindrical body abating buildup of negative pressure that would otherwise draw a gastric contents from the enteral tube into the syringe, thus allowing the syringe to remain connected to the syringe port while the syringe plunger is removed to refill the syringe with a treatment contents during patient treatment.

2. The improved enteral connector in accordance with claim 1, further comprises:
a valve port cover is removable and when secured seals the valve port closed preventing air from ingressing into the cylindrical body through the one-way air valve.

3. The improved enteral connector in accordance with claim 2, the user rotates the columnar plug to select a syringe port block position where at least one of the flow hole is aligned with the proximal port, the distal port, and the valve port effectuating fluid communication between the proximal port and the distal port, the syringe port is absent one of the flow hole and blocked from fluid communication, in treatment when the suction line is interconnected with the proximal port the user secures the valve port cover to the valve port preventing air from ingressing into the cylindrical body to maintain suction within the cylindrical body during treatment.

4. The improved enteral connector in accordance with claim 1, the user rotates the columnar plug to select a distal port block position where at least one of the flow hole is aligned with each of the syringe port, the proximal port, and the valve port effectuating fluid communication of liquid between the syringe port and the proximal port, and fluid communication of air between the valve port and the syringe port, the distal port is absent one of the flow hole and blocked from fluid communication, the user secures the syringe to the syringe port, during removal of the syringe plunger from the syringe air ingresses through the one-way air valve into the cylindrical body abating buildup of negative pressure that would draw a feeding bag tube contents from the feeding bag tube into the syringe, thus allowing the syringe to remain connected to the syringe port while the syringe plunger is removed to refill the syringe with the treatment contents during patient treatment.

5. The improved enteral connector in accordance with claim 1, each of the valve port, proximal port, syringe port, and distal port are positioned at 90-degree increments around circumference of the cylindrical body.

6. The improved enteral connector in accordance with claim 1, each of the proximal port, distal port, and syringe port are ENFIT compatible.

7. The improved enteral connector in accordance with claim 1, further comprising:
a T-shaped handle that comprises a plurality of protrusions, when the columnar plug is rotated to the proximal port block position the plurality of protrusions are aligned with the valve port, proximal port, distal port, and the syringe port, the plurality of protrusions comprise indicia that in the proximal port block position indicate the proximal port is off or blocked and the distal port, the syringe port, and the valve port are open or unblocked.

8. The improved enteral connector in accordance with claim 7, the T-shaped handle is integrally formed on the columnar plug.

9. The improved enteral connector in accordance with claim 1, further comprising:
a syringe port cover is removable and when secured seals the syringe port closed.

10. An improved enteral connector comprising:
a cylindrical body;
a valve port interconnected on circumference of the cylindrical body;
a one-way air valve interconnects with the valve port and is configured to allow ambient air to ingress into the cylindrical body through the one-way air valve abating buildup of negative pressure within the cylindrical valve body;
a valve port cover is removable and when secured seals the valve port closed preventing air from ingressing into the cylindrical body through the one-way air valve;
a proximal port is interconnected on circumference of the cylindrical body, the proximal port is configured to interconnect with a feeding bag tube or a suction line;
a distal port is interconnected on circumference of the cylindrical body, the distal port is configured to interconnect with an enteral tube;
a syringe port is interconnected on circumference of the cylindrical body, the syringe port is configured to interconnect with a syringe, the syringe comprises a syringe plunger that is removable from the syringe; and
a columnar plug interconnects with the cylindrical body and is rotatable to at least a proximal port block position, the columnar plug having more than one of a flow hole that are interconnected, each of the flow hole is perpendicular to outer circumference of the columnar plug and extends inward interconnecting with other of the flow hole proximate to center of interior of the columnar plug, each of the flow hole are positioned on circumference of the columnar plug in manner that when the columnar plug is rotated by a user, to each block position, fluid communication of air or liquid is allowed to pass through the flow holes between fewer than all of the following: the valve port, the proximal port, the distal port, and the syringe port, the user rotates the columnar plug to select the proximal port block position where at least one of the flow hole is aligned with each of the syringe port, the distal port, and the valve port effectuating fluid communication of liquid between the syringe port and the distal port, and fluid communication of air between the valve port and the syringe port, the proximal port is absent one of the flow hole and blocked from fluid communication, in operation the user secures the syringe to the syringe port, during removal of the syringe plunger from the syringe air ingresses through the one-way air valve into the cylindrical body abating buildup of negative pressure that would otherwise draw a gastric contents from the enteral tube into the syringe, thus allowing the syringe to remain connected to the syringe port while the syringe plunger is removed to refill the syringe with a treatment contents during patient treatment.

11. The improved enteral connector in accordance with claim 10, each of the valve port, proximal port, syringe port, and distal port are positioned at 90-degree increments around circumference of the cylindrical body.

12. The improved enteral connector in accordance with claim 10, each of the proximal port, distal port, and syringe port are ENFIT compatible.

13. The improved enteral connector in accordance with claim 10, the user rotates the columnar plug to select a syringe port block position where at least one of the flow hole is aligned with the proximal port, the distal port, and the valve port effectuating fluid communication between the proximal port and the distal port, the syringe port is absent one of the flow hole and blocked from fluid communication, in treatments where the suction line is interconnected with the proximal port the user secures the valve port cover to the valve port preventing air from ingressing into the cylindrical body to maintain suction within the cylindrical body during treatment.

14. The improved enteral connector in accordance with claim 10, the user rotates the columnar plug to select a distal port block position where at least one of the flow hole is aligned with each of the syringe port, the proximal port, and the valve port effectuating fluid communication of liquid between the syringe port and the proximal port, and fluid communication of air between the valve port and the syringe port, the distal port is absent one of the flow hole and blocked from fluid communication, the user secures the syringe to the syringe port, during removal of the syringe plunger from the syringe air ingresses through the one-way air valve into the cylindrical body abating buildup of negative pressure that would otherwise draw a feeding bag tube contents from the feeding bag tube into the syringe, thus allowing the syringe to remain connected to the syringe port while the syringe plunger is removed to refill the syringe with the treatment contents during patient treatment.

15. The improved enteral connector in accordance with claim 10, further comprising:
a T-shaped handle that comprises a plurality of protrusions, when the columnar plug is rotated to the proximal port block position the plurality of protrusions are aligned with the valve port, proximal port, distal port, and the syringe port, the plurality of protrusions comprise indicia that in the proximal port block position indicating the proximal port is off or blocked and the distal port, the syringe port, and the valve port are open or unblocked.

16. The improved enteral connector in accordance with claim 15, the T-shaped handle is integrally formed on the columnar plug.

17. The improved enteral connector in accordance with claim 10, further comprising:
a syringe port cover is removable and when secured seals the syringe port closed.

18. A method of using an improved enteral connector, the method comprising the steps of:
attaching an enteral tube to a distal port of an improved enteral connector, the improved enteral connector comprising a cylindrical body that comprises a valve port, a proximal port, the distal port, and a syringe port, the proximal port is configured to interconnect with a feeding bag tube or a suction line, a one-way air valve interconnects with the valve port and is configured to allow ambient air to ingress into the cylindrical body through the one-way air valve abating buildup of negative pressure within the cylindrical valve body, a columnar plug interconnects with the cylindrical body and is rotatable to at least a proximal port block position, the columnar plug having more than one of a flow hole that are interconnected, each of the flow hole is perpendicular to outer circumference of the columnar plug and extends inward interconnecting with other of the flow hole proximate to center of interior of the columnar plug, each of the flow hole are positioned on circumference of the columnar plug in manner that when the columnar plug is rotated by a user, to each block position, fluid communication of air or liquid is allowed to pass through the flow holes between fewer than all of the following: the valve port, the proximal port, the distal port, and the syringe port;
connecting a syringe to the syringe port, the syringe comprises a syringe plunger that is removable from the syringe;
setting, by way of the user, the proximal port block position by rotating the columnar plug, where at least one of the flow hole is aligned with each of the syringe port, the distal port, and the valve port effectuating fluid communication of liquid between the syringe port and the distal port, and fluid communication of air between the valve port and the syringe port, the proximal port is absent one of the flow hole and blocked from fluid communication;
dispensing a treatment contents from the syringe through the improved enteral connector and into the enteral tube;
removing the syringe plunger while the syringe is connected to the syringe port, during removal of the syringe plunger from the syringe air ingresses through the one-way air valve into the cylindrical body abating buildup of negative pressure that would draw a gastric contents from the enteral tube into the syringe, thus allowing the syringe to remain connected to the syringe port while the syringe plunger is removed; and
refilling the syringe with the treatment contents, and then returning to the step of dispensing until treatment is complete.

19. The method in accordance with claim 19, further comprising the step of:
setting, by way of the user, a syringe port block position by rotating the columnar plug, where at least one of the flow hole is aligned with the proximal port, the distal port, and the valve port effectuating fluid communication between the proximal port and the distal port, the syringe port is absent one of the flow hole and blocked from fluid communication, in treatment when the suction line is interconnected with the proximal port the user secures a valve port cover to the valve port preventing air from ingressing into the cylindrical body to maintain suction within the cylindrical body during treatment.

20. The method in accordance with claim 18, further comprising the step of:
setting, by way of the user, a distal port block position by rotating the columnar plug, where at least one of the flow hole is aligned with each of the syringe port, the proximal port, and the valve port effectuating fluid communication of liquid between the syringe port and the proximal port, and fluid communication of air between the valve port and the syringe port, the distal port is absent one of the flow hole and blocked from fluid communication, the user secures the syringe to the syringe port, during removal of the syringe plunger from the syringe air ingresses through the one-way air valve into the cylindrical body abating buildup of negative pressure that would otherwise draw a feeding bag tube contents from the feeding bag tube into the syringe, thus allowing the syringe to remain connected to the syringe port while the syringe plunger is removed to refill the syringe with the treatment contents during patient treatment.
